(19) United States
(12) Patent Application Publication
(10) Pub. No.: US 2006/0271133 A1
(43) Pub. Date: Nov. 30, 2006

(54) Cryotherapeutic device

(76) Inventor: Aleksandrs Baranovs, Riga (LV)

Correspondence Address: OLSON & HIERL, LTD., 20 NORTH WACKER DRIVE, 36TH FLOOR, CHICAGO, IL, 60606, US

(21) Appl. No.: 10/553,333

(22) PCT Filed: Apr. 16, 2004
(86) PCT No.: PCT/LV04/00003

§ 371 Date: Oct. 13, 2005

(30) Foreign Application Priority Data
Apr. 16, 2003 (LV) P-03-41

(51) Int. Cl. A61F 7/00; A61F 7/12

(52) U.S. Cl. 607/96

(57) ABSTRACT

The discovery refers to medical equipment, but more specifically is used as a facility for rehabilitation and prophylactic physiotherapeutic procedures. The discovery is aimed at reduction of liquid nitrogen used during procedures and improvement of working conditions for servicing personnel.-This aim is reached due to the fact that the cryotherapeutic facility, which includes a subsequently installed cabin for the patient, the heat-isolating reservoir with the ventilator and the system of cryostatistics with gas consumption stimulator is supplemented with a system installed for utilization of produced gas, which consists of pipes with a tap and a three-step valve. Besides, the gas stimulator is linked to the pipe with heat-isolating reservoir, but the ventilator is installed on the pipe, which connects the cabin for the patient with the heat-isolating reservoir. Repeated use of low-temperature gas reduces consumption of liquid nitrogen by 2.5 times. Existence of gas in the facility of utilization system does not allow movement of nitrogen vapor into the room of medical procedures. The system automatically removes all excess gas produced as a result of vaporization of liquid nitrogen.

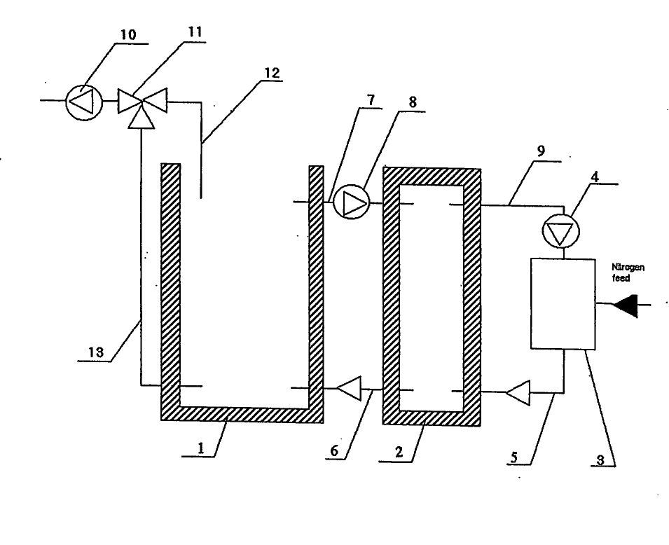

The discovery refers to medical equipment, but more specifically is used as a facility for rehabilitation and prophylactic physiotherapeutic procedures.

This equipment is well-known for general cryotherapy and consists of a chamber for locating of a patient, the system for drying, cooling and transportation of cold air. The equipment is also furnished with heat-isolating reservoir with a ventilator, which is connected to the chamber for locating the patient and the system of drying, cooling and transportation of air. Besides, there is a shutoff valve, which is located between the chamber for locating the patient and the heat-isolating reservoir. /prototype-author's registration No. 1684979 in the USSR/ A disadvantage of this discovery is increased utilization of liquid nitrogen, which is connected with the fact that the cryogenic gas after the chamber for locating the patient is discharged in atmosphere. Usage of nitrogen means high energy consumption for carrying out medical procedures.

The proposed useful model is aimed at reducing energy expenses and improvement of work safety for medical personnel.

This aim can be reached based on the fact that the cryotherapeutic facility, which includes a subsequently installed cabin for the patient, the heat-isolating reservoir with the ventilator and the system of cryostatistics with gas consumption stimulator is supplemented with a system installed for utilization of produced gas, which consists of pipes with a tap and a three-step valve. Besides, the gas stimulator is linked to the pipe with heat-isolating reservoir, but the ventilator is installed on the pipe, which connects the cabin for the patient with the heat-isolating reservoir.

Reduction of electricity is achieved by means of the method that the system of cryostatistics is enforced by being thermally closed because the gas stimulator is connected with the pipe of heat-isolating reservoir, but the ventilator is installed on the pipe, which connects the cabin for locating the patient with the heat-isolating reservoir. This gives a possibility to decrease electricity consumption at the expense of circularization of processed gas through the system of cryostatistics, which cools gas to the indicated temperature of cryo-operation and ensures equalizing of field of temperatures in the cabin for locating the patient. Utilization of processed gas after all is based on consumption of liquid nitrogen.

Improvement of work safety for medical personnel which services cryotherapeutic equipment is achieved due to the fact that it additionally includes a system for reducing of excess gas a result of processing, which is done by pipes with a tap and a three-step valve. This gives a possibility to remove from the upper part of the cabin for locating the patient the excess gas into the atmosphere, i.e., the steam of liquid nitrogen does not get into the room where procedures are carried out. The chain of cryotherapeutic equipment is seen in the enclosed picture. It consists of subsequently connected cabin 1 for locating the patient, heat-isolating reservoir 2, system for cryostatistics 3. These devices are linked to each other with pipes: pipe 5 connects the system for cryostatistics 3 with the heat-isolating reservoir 2, pipe 6 connects the heat-isolating reservoir 2 with the cabin for locating the patient 1; pipe 7 with the ventilator 8 connects at the upper part of facility the cabin for locating the patient 1 with heat-isolating reservoir 2, pipe 9 with the gas stimulator 4 connects the heat-isolating reservoir 2 with the system for cryostatistics 3. The cryotherapeutic facility also includes a system for removal of processed gas, which consists of a pipe for final pumping 13 with the ventilator 10, three-step valve 11 and pumping pipe 12.

The facility is working in the following way. The cold gas from the heat-isolating reservoir 2 gets into the cabin 1 for locating the patient through the pipe 6. When going upward, gas takes the heat from the body of patient and walls of the cabin 1 and warms up by 10-20 degrees. At the upper part of the cabin, there is a pipe 7, on which a ventilator 8 is installed, which ensures circularization of gas between the cabin for locating the patient 1 and the heat-isolating reservoir 2. Temperature of gas in the heat-isolating reservoir 2 is maintained by means of system for cryostatistics 3. Then, gas through the pipe 9 warms up by gas stimulator 4 into the system for cryostatistics 3 where it is cooled by means of heat exchange with liquid nitrogen and through the pipe 5 it returns to the heat-isolating reservoir 2.

During a procedure in the facility, vaporization of liquid nitrogen in the system for cryostatistics 3 results in excess gas in the amount of 3-4 kgs, and gas through the pipe 12 and three-step valve 11 is pumped from the level which is located between the pipe 7 and the upper part of cabin 1. Removal of nitrogen gas in the form of vapor protects breathing organs of the patient and limits entry of nitrogen vapor into the room of procedures.

At the end of the procedure of therapeutic impact, three-step valve 11 gets connected to the pipe for removal of processed gas 10. Low-temperature gas in the amount of 1-1.2 kgs is removed into the atmosphere before the patient gets out of the cabin, therefore it cannot get into the room of procedures.

Operation of cryotherapeutic facility has shown that the given construction is energy-saving and simple in manufacturing and it will find a good realization in medicine.

1. The cryotherapeutic facility, which consists of the cabin for locating the patient, the heat-isolating reservoir with a ventilator and a system for cryostatistics with heat-carrier consumption stimulator, is different from others with the fact that it additionally includes a system of utilization of processed gas which is performed by pipes with the ventilator of three-step valve, heat-carrier consumption stimulator is connected by means of a pipe with the heat-isolating reservoir, but the ventilator is installed on the pipe, which connects the cabin for locating the patient with the heat-isolating reservoir.
